(19) United States
(12) Patent Application Publication
(10) Pub. No.: US 2020/0368377 A1
(43) Pub. Date: Nov. 26, 2020

(54) DEVICE AND METHODS TO DESTROY BACTERIA, MOLDS, FUNGI AND VIRUSES AND FOR REDUCING INFLAMMATION AND MARKERS IN ORGANS AND TISSUE AND TO EXTEND THE UTILITY OF ANTIBIOTICS

(71) Applicant: Tissue Regeneration Technologies, LLC, Woodstock, GA (US)

(72) Inventor: John F. Warlick, Woodstock, GA (US)

(21) Appl. No.: 16/879,979

(22) Filed: May 21, 2020

Related U.S. Application Data
(60) Provisional application No. 62/852,683, filed on May 24, 2019.

(51) Int. Cl. A61L 2/02; A61K 41/00

(52) U.S. Cl. CPC A61L 2/02; A61K 45/06; A61K 41/00

(57) ABSTRACT

A method of treating a patient having inflammation or an infection from bacteria or molds or fungi or virus by destroying bacteria or molds or fungi or virus has the step of directing one or more sound wave treatments into the patient to destroy bacteria or molds or fungi or virus. The sound wave treatments cause an improved blood supply, a disruption of cellular membranes and a cellular communication causing the patient's cells to identify and attack the bacteria, mold fungi or virus and further causes recruiting or stimulating an increase in anti-microbial peptides. The method further can have the step of administering medications to the patient including, but not limited to anti-viral medications, antibiotics, anti-fungal medications or anti-mold medications, wherein the sound wave treatment extends the useful life of the medications.

### FIELD OF THE INVENTION

The present invention relates to the use of sound waves, more particularly acoustic shock waves to destroy bacteria, molds, fungi and viruses and for reducing inflammation and markers in organs and tissue and to extend the utility of antibiotics to eradicate infections and to methods to prevent infections.

### BACKGROUND OF THE INVENTION

Almost all living creatures including plants are formed of cellular tissues. In virtually every living being these cellular communities form an outer protective barrier of tissues. In mammals this protective barrier is commonly referred to as skin. Similarly, in vegetables and plants the outer shell is really a protective barrier of skin or a peel that grows as the vegetable or fruit matures providing a shield from intrusions to the underlying and generally more vulnerable inner tissue. For example, in citrus fruits the juicy high liquid content of these tissues would be impossible to mature without the protective outer peel.

Accordingly, the use of such natural shields or barriers to protect more vulnerable cells or tissue is the norm.

It is therefore of little surprise that on the molecular level bacteria whether aerobic or anaerobic have generally been known to exhibit an outer protective cellular membrane similar to a skin and any treatment to destroy such a bacteria typically required weakening or penetrating this outer membrane. Once penetration occurred the viability of the organism was diminished resulting in a cessation of viability.

Bacteria while being a relatively lower order entity has nonetheless a very strong and evolutionary desire to survive and thus is one of the more adaptive organisms found on earth. Mutant strains of bacteria are commonly feared because of their huge capacity to adapt to threats particularly those involving the use of microbial disinfectants and antibiotics used to fight disease.

Microorganisms grow through a form of cellular division. Blood agar cultures are used to grow colonies of bacteria. The cluster starts out invisible to the naked eye and within 24 to 48 hours can be a large colony of millions of bacteria. This has always been a well known phenomenon of bacterial growth.

Almost all of the prior art literature on the subject of eliminating or preventing bacterial or viral infections suggests one or more drugs or chemical agents as the solution to this problem.

What is sorely lacking are safe and reliable devices and methods to break down the cellular barrier properties of these complex molds, fungi and microbial or viral infections to reduce their resistance to disinfectants and antibiotics.

It is therefore an object of the present invention to provide such a method to reduce or eradicate infections not only on surfaces, but within tissues and organs.

It is a further objective to enhance the use of medications to better attack and destroy the infections without losing effectiveness due to mutations of the disease becoming resistant to the medications like antibiotics.

### SUMMARY OF THE INVENTION

A method of treating a patient having an infection from bacteria or molds or fungi or virus by destroying bacteria or molds or fungi or virus has the step of directing one or more sound wave treatments into the patient to destroy bacteria or molds or fungi or virus. The sound wave treatments cause an improved blood supply, a disruption of cellular membranes and a cellular communication causing the patient's cells to identify and attack the bacteria, mold fungi or virus and further causes recruiting or stimulating an increase in anti-microbial peptides. The method further can have the step of administering medications to the patient including, but not limited to anti-viral medications, antibiotics, anti-fungal medications or anti-mold medications, wherein the sound wave treatment extends the useful life of the medications.

The sound wave treatments increase the permeability of the patient's cell membranes allowing an increase in releasing anti-microbial peptides and inflow of the medications into the cells while increasing the blood supply toward the infection. The sound wave treatment can be provided either prior to, during or after administering medications or any combination thereof. The infection's resistance to medications is reduced by the sound wave treatments and the medications effectiveness against an infection is enhanced by the sound wave treatments. The dosages or strength of the medications can be reduced when used in combination with the sound wave treatments. The sound waves are acoustic shock waves. The acoustic shock waves can be focused or non-focused, convergent, divergent, planar or nearly planar, radial or spherical, shaped or otherwise reflected. The sound wave treatments are emitted by a generator. The generator can be one of a radial, a spherical, a ballistic, a linear, a piezoelectric, or an electrohydraulic generator. The sound wave treatments can be administered with or without cavitation and can be administered with or without some cellular destruction and with or without a sensation of pain.

In one embodiment, the present invention uses pressure pulse or acoustic shock waves as a means to treat a patient diagnosed with inflammation or one or more infections of a microbial or viral source or other sources from mold or fungi. The infections causing at least localized inflammation. The method has the steps of locating a region or location of the inflammation or infection, activating a pressure pulse or acoustic shock wave generating source and emitting pressure pulse or acoustic waves and directing the pulses or shock waves to impinge the inflammation directly or by indirectly impinging a reflexology zone to destroy, fracture, fragment or otherwise open the microbial or viral source to eradicate the source and reduce the inflammation.

The present invention provides a means for fracturing and breaking the outer organisms barrier into fragments that can be absorbed or flushed away by healthy cells of the patient as well as exposing the microbial or viral organisms for eradication.

The present invention provides a germicidal energy that alone or in combination with stimulated healthy cells of the patient destroys the microbial or viral sourced infection.

The present invention can be used alone or in combination with surgical procedures or with drugs or antibiotics or other forms of medicaments to enhance the effectiveness of the drugs, antibiotics or medicaments as a result of increasing blood supply, cellular perfusion of host cell membranes and penetration of biofilms to eradicate the infection.

In one embodiment of the invention the present method is used as a pre-occurrence preventative treatment prior to the formation of an infection for patients of high risk for the occurrence.

In other embodiments the method is used post-occurrence for the treatment of infections found in or on tissues or organs such as the urinary tract, the reproductive organs, the heart, more particularly heart valves, heart pumps or other implants such as artificial joints, pacemakers or medical hardware such as screws, rods or pins with deposits of one or more infections.

As used throughout the invention the patient is considered to be any infection supporting system or being. In mammals, the being may be an animal or a human. The system may be any system be it mechanical or living. In living systems, it may be the cardiovascular system, the urological or the reproductive system, the digestive system, the neurological, the periodontal region of teeth and gums or any tissue or organ found in the patient of an infection or at risk or candidate patient.

### Definitions

“aerobic” living, active, or occurring only in the presence of oxygen.

“anaerobic” living, active, or occurring in the absence of free oxygen.

“apoptosis” is the biological process of controlled, programmed cell death, by means of which cells die by a process of condensation without the release of cell contents into the surrounding milieu.

A “curved emitter” is an emitter having a curved reflecting (or focusing) or emitting surface and includes, but is not limited to, emitters having ellipsoidal, parabolic, quasi parabolic (general paraboloid) or spherical reflector/reflecting or emitting elements. Curved emitters having a curved reflecting or focusing element generally produce waves having focused wave fronts, while curved emitters having a curved emitting surfaces generally produce wave having divergent wave fronts.

“cystic fibrosis” a common disease especially in Caucasian populations that appears usually in early childhood, is inherited as a recessive monogenic trait, involves functional disorder of the exocrine glands, and is marked especially by faulty digestion due to a deficiency of pancreatic enzymes, by difficulty in breathing due to mucus accumulation in airways, and by excessive loss of salt in the sweat.

“cytoplasm” The part of a cell that contains the CYTOSOL and small structures excluding the CELL NUCLEUS; MITOCHONDRIA; and large VACUOLES.

“Divergent waves” in the context of the present invention are all waves which are not focused and are not plane or nearly plane. Divergent waves also include waves which only seem to have a focus or source from which the waves are transmitted. The wave fronts of divergent waves have divergent characteristics. Divergent waves can be created in many different ways, for example: A focused wave will become divergent once it has passed through the focal point. Spherical waves are also included in this definition of divergent waves and have wave fronts with divergent characteristics.

“endocarditis” inflammation of the lining of the heart and its valves.

“extracorporeal” occurring or based outside the living body.

A “generalized paraboloid” according to the present invention is also a three-dimensional bowl. In two dimensions (in Cartesian coordinates, x and y) the formula yⁿ=2px [with n being ≠2, but being greater than about 1.2 and smaller than 2, or greater than 2 but smaller than about 2.8]. In a generalized paraboloid, the characteristics of the wave fronts created by electrodes located within the generalized paraboloid may be corrected by the selection of (p (−z,+z)), with z being a measure for the burn down of an electrode, and n, so that phenomena including, but not limited to, burn down of the tip of an electrode (−z,+z) and/or disturbances caused by diffraction at the aperture of the paraboloid are compensated for.

“lactate dehydrogenase (LDH)” A tetrameric enzyme that, along with the coenzyme NAD+, catalyzes the interconversion of lactate and pyruvate. In vertebrates, genes for three different subunits (LDH-A, LDH-B and LDH-C) exist.

“mitochondria” Semiautonomous, self-reproducing organelles that occur in the cytoplasm of all cells of most, but not all, eukaryotes. Each mitochondrion is surrounded by a double limiting membrane. The inner membrane is highly invaginated, and its projections are called cristae. Mitochondria are the sites of the reactions of oxidative phosphorylation, which result in the formation of ATP. They contain distinctive RIBOSOMES, transfer RNAs (RNA, TRANSFER); AMINO ACYL T RNA SYNTHETASES; and elongation and termination factors. Mitochondria depend upon genes within the nucleus of the cells in which they reside for many essential messenger RNAs (RNA, MESSENGER). Mitochondria are believed to have arisen from aerobic bacteria that established a symbiotic relationship with primitive protoeukaryotes.

“necrosis” A pathological process caused by the progressive degradative action of enzymes that is generally associated with severe cellular trauma. It is characterized by mitochondrial swelling, nuclear flocculation, uncontrolled cell lysis, and ultimately CELL DEATH.

A “paraboloid” according to the present invention is a three-dimensional reflecting bowl. In two dimensions (in Cartesian coordinates, x and y) the formula y²=2px, wherein p/2 is the distance of the focal point of the paraboloid from its apex, defines the paraboloid. Rotation of the two-dimensional figure defined by this formula around its longitudinal axis generates a de facto paraboloid.

“phagocytosis” The engulfing of microorganisms, other cells, and foreign particles by phagocytic cells.

“Plane waves” are sometimes also called flat or even waves. Their wave fronts have plane characteristics (also called even or parallel characteristics). The amplitude in a wave front is constant and the “curvature” is flat (that is why these waves are sometimes called flat waves). Plane waves do not have a focus to which their fronts move (focused) or from which the fronts are emitted (divergent). “Nearly plane waves” also do not have a focus to which their fronts move (focused) or from which the fronts are emitted (divergent). The amplitude of their wave fronts (having “nearly plane” characteristics) is approximating the constancy of plain waves. “Nearly plane” waves can be emitted by generators having pressure pulse/shock wave generating elements with flat emitters or curved emitters. Curved emitters may comprise a generalized paraboloid that allows waves having nearly plane characteristics to be emitted.

A “pressure pulse” according to the present invention is an acoustic pulse which includes several cycles of positive and negative pressure. The amplitude of the positive part of such a cycle should be above about 0.1 MPa and its time duration is from below a microsecond to about a second. Rise times of the positive part of the first pressure cycle may be in the range of nano-seconds (ns) up to some milli-seconds (ms). Very fast pressure pulses are called shock waves. Shock waves used in medical applications do have amplitudes above 0.1 MPa and rise times of the amplitude can be below 1000 ns, preferably at or below 100 ns. The duration of a shock wave is typically below 1-3 micro-seconds (ρs) for the positive part of a cycle and typically above some micro-seconds for the negative part of a cycle.

Prostate-specific antigen, also known as gamma-seminoprotein or kallikrein-3, is a glycoprotein enzyme encoded in humans by the KLK3 gene. PSA is a member of the kallikrein-related peptidase family and is secreted by the epithelial cells of the prostate gland. PSA is present in small quantities in the serum of men with healthy prostates, but is often elevated in the presence of prostate cancer or other prostate disorders. PSA is not a unique indicator of prostate cancer, but may also detect prostatitis or benign prostatic hyperplasia. The PSA test is a blood test used primarily to screen for prostate cancer. The test measures the amount of prostate-specific antigen (PSA) in your blood. PSA is a protein produced by both cancerous and noncancerous tissue in the prostate, a small gland that sits below the bladder in men.

“Reflexology zone” as used herein means an area or pressure point on the feet or hands that are access pathways to every organ, gland, muscle, etc. These pathways between pressure points and other parts of the body are thought to be connected via the nervous system and that a neurological relationship exists between the skin and the internal organs, and that the whole nervous system adjusts to a stimulus. According to reflexology theory, application of pressure to feet, hands, or ears sends a calming message from the peripheral nerves in these extremities to the central nervous system, which in turn signals the body to adjust the tension level. This enhances overall relaxation, removes stress, brings internal organs and their systems into a state of optimum functioning, and increases blood supply which brings additional oxygen and nutrients to cells and enhances waste removal. It positively affects the circulatory, respiratory, endocrine, immune, and neuropeptide systems in the body.

“Shock Wave”: As used herein is defined by Camilo Perez, Hong Chen, and Thomas J. Matula; Center for Industrial and Medical Ultrasound, Applied Physics Laboratory, University of Washington, 1013 NE 40th Street, Seattle, Wash. 98105; Maria Karzova and Vera A. Khokhlovab; Department of Acoustics, Faculty of Physics, Moscow State University, Moscow 119991, Russia; (Received 9 Oct. 2012; revised 16 Apr. 2013; accepted 1 May 2013) in their publication, “Acoustic field characterization of the Duolith: Measurements and modeling of a clinical shock wave therapy device”; incorporated by reference herein in its entirety.

Wave energy or energy flux density: the measurement of energy flux density is defined as the energy directed toward the target or region being treated. This is not energy at the gap between electrodes, but rather the energy transmitted toward the patient's tissue through the skin. Important to distinguish that the energy levels discussed pertain to the energy delivered to the targeted tissues and not at the discharge point between the electrode tips. Spherical waves have a huge amount of energy produced between the tips to deliver adequate energy to the targeted tissues since they do not have the advantage of a lens.

Waves/wave fronts described as being “focused” or “having focusing characteristics” means in the context of the present invention that the respective waves or wave fronts are traveling and increase their amplitude in direction of the focal point. Per definition the energy of the wave will be at a maximum in the focal point or, if there is a focal shift in this point, the energy is at a maximum near the geometrical focal point. Both the maximum energy and the maximal pressure amplitude may be used to define the focal point.

### BRIEF DESCRIPTION OF THE DRAWINGS

The invention will be described by way of example and with reference to the accompanying drawings in which:

FIG. 1 is a simplified depiction of a pressure pulse/shock wave (PP/SW) generator with focusing wave characteristics.

FIG. 2 is a simplified depiction of a pressure pulse/shock wave generator with plane wave characteristics.

FIG. 3 is a simplified depiction of a pressure pulse/shock wave generator with divergent wave characteristics.

FIG. 4a is a simplified depiction of a pressure pulse/shock wave generator having a focusing element in the form of an ellipsoid. The waves generated are focused.

FIG. 4b is a simplified depiction of a pressure pulse/shock wave generator having a parabolic reflector element and generating waves that are disturbed plane.

FIG. 4c is a simplified depiction of a pressure pulse/shock wave generator having a quasi parabolic reflector element (generalized paraboloid) and generating waves that are nearly plane/have nearly plane characteristics.

FIG. 4d is a simplified graphic depiction of a generalized paraboloid with better focusing characteristic than a paraboloid in which n=2. The electrode usage is shown. The generalized paraboloid, which is an interpolation (optimization) between two optimized paraboloids for a new electrode and for a used (burned down) electrode is also shown.

FIG. 5 is a simplified depiction of a pressure pulse/shock wave generator being connected to a control/power supply unit.

FIG. 6 is a simplified depiction of a pressure pulse/shock wave generator comprising a flat EMSE (electromagnetic shock wave emitter) coil system to generate nearly plane waves as well as an acoustic lens. Convergent wave fronts are leaving the housing via an exit window.

FIG. 7 is a simplified depiction of a pressure pulse/shock wave generator having a flat EMSE coil system to generate nearly plane waves. The generator has no reflecting or focusing element. As a result, the pressure pulse/shock waves are leaving the housing via the exit window unfocused having nearly plane wave characteristics.

FIG. 8 is a simplified depiction of a pressure pulse/shock wave generator having a flat piezoceramic plate equipped with a single or numerous individual piezoceramic elements to generate plane waves without a reflecting or focusing element. As a result, the pressure pulse/shock waves are leaving the housing via the exit window unfocused having nearly plane wave characteristics.

FIG. 9 is a simplified depiction of a pressure pulse/shock wave generator having a cylindrical EMSE system and a triangular shaped reflecting element to generate plane waves. As a result, the pressure pulse/shock waves are leaving the housing via the exit window unfocused having nearly plane wave characteristics.

FIG. 10 shows an exemplary shock wave generator device.

FIG. 11 shows the shock wave generator device directed at a reflexology zone on a foot of a patient.

FIG. 12 shows the shock wave generator device directed at a reflexology zone on a hand of a patient.

FIGS. 13-13C show schematic views showing general reflexology locations of the foot and ankle area in the human body.

FIG. 14 shows a schematic view showing general reflexology locations of the hand in the human body.

### DETAILED DESCRIPTION OF THE INVENTION

In the extracorporeal shock wave or pressure pulse method of treating an infection of a patient, the administered shock waves or pressure pulses are directed to a treatment location or target site on the anatomy. In this invention, the term target site refers to either a location near the source of the infection or to a reflexology location for a specific orthopedic bone structure, nerve, gland and the tissue of the hand or foot at the desired reflexology zone or region being in the path of the shock wave applicator. As used herein, “near” recognizes that the emitted shock waves or pressure pulses are transmitted through the skin and subcutaneous tissue directed toward the treatment location, preferably at or in close proximity to the treatment location or site. The patient is placed in a convenient orientation to permit the source of the emitted waves to most directly send the waves to the target site to initiate shock wave stimulation of the target area. Assuming the target area is within a projected area of the wave transmission, a single transmission dosage of wave energy may be used. The transmission dosage can be from a few seconds to 20 minutes or more dependent on the condition. Preferably the waves are generated from an unfocused or focused source. The unfocused waves can be divergent or near planar and having a low-pressure amplitude and density in the range of 0.00001 mJ/mm² to 1.0 mJ/mm² or less, most typically below 0.2 mJ/mm². These are typically generated by spherical or radial wave generators, ballistic or electrohydraulic wave or piezoelectric shock wave generators. The focused source can use a focused beam of waves or can optionally use a diffusing lens or have a far-sight focus to minimize if not eliminate having the localized focus zone within the tissue. Preferably the focused shock waves are used at a similarly effective low energy transmission or alternatively can be at higher energy but wherein the tissue target site is disposed pre-convergence inward of the geometric focal point of the emitted wave transmission. Understanding the higher the energy used, the more sensation of pain the patient may experience. In these cases, cavitation can and often does occur as well as bruising and come cell damage.

These shock wave energy transmissions are effective in stimulating a cellular response and in some cases, such as unfocused low energy, and even low energy focused emissions can be accomplished without creating the localized hemorrhaging caused by rupturing cavitation bubbles in the tissue of the target site. This effectively ensures the patient does not have to experience the sensation of pain so common in the higher energy focused wave forms having a focal point at or within the targeted treatment site. Higher energy acoustic shock waves or pressure pulses including focused waves can be used.

The target site within the body may be such that the patient or the generating source must be reoriented relative to the site and a second, third or more treatment dosage can be administered. At a low energy, the common problem of localized hemorrhaging is reduced making it more practical to administer multiple dosages of waves from various orientations to further optimize the treatment and cellular stimulation of the target site. Alternatively, focused high energy multiple treatments can be equally effective, but with induced pain and more discomfort to the patient. The use of low energy focused or un-focused waves at the target site enables multiple sequential treatments. Alternatively, the wave source generators may be deployed in an array wherein the subject patient is effectively enveloped or surrounded by a plurality of low energy wave source generators which can be simultaneously bombarding the target site from multiple directions. Such arrays include linear type devices.

The goal in such treatments is to provide 100 to 3000 acoustic shock waves or pressure pulses. Typically, at a voltage of 14 kV to 28 kV across a spark gap generator in a single treatment preferably or one or more adjuvant treatments by targeting the site directly by impinging the emitted waves toward the infection or indirectly on the desired reflexology target.

The present method, in many cases, does not rely on precise site location per se. The physician's general understanding of the anatomy of the patient should be sufficient to locate a desirable direct path or using a reflexology target site to indirectly attack the infection be treated. The treated area can withstand a far greater number of shock waves based on the selected energy level being emitted. For example, at very low energy levels the stimulation exposure can be provided over prolonged periods as much as 20 minutes if so desired. At higher energy levels the treatment duration can be shortened to less than a minute, less than a second if so desired. The selected treatment dosage can include the avoidance or minimization of cell hemorrhaging and other kinds of damage to the cells or tissue while still providing a stimulating cellular release activation of upregulation of the antimicrobial peptide LL37, a protein that can bind with RNA to destroy the infection, and also VEGF and other growth factors and can also be used to modulate and regulate hormonal secretions from a specific targeted gland by emitting waves to a desired direct path or indirectly selected using a targeted reflexology zone. In other cases where the precise location must be known, the use of an applicator acoustic wave emission is directed by an ultrasound image, preferably the applicator has a software program coupled to the imaging device to allow the doctor to visualize the area being treated. The applicator can be hand held or manipulated in a fixture, if so desired, in either way the doctor can see the reflexology zone for any gland to be stimulated and the selected reflexology zone reflects the path of the wave transmission to modulate that bone structure, nerve or gland.

A key advantage of the present inventive methodology is that it is complimentary to conventional medical procedures. In the case of any other procedure, the area of the patient can be post operatively bombarded with sound waves to stimulate cellular release of healing agents and growth factors. Most preferably such patients may be provided more than one such ESWT treatment with an intervening dwell time for cellular relaxation prior to secondary and tertiary treatments.

The underlying principle of these sound wave therapy methods is to stimulate the body's own natural healing capability. This is accomplished by deploying shock waves to stimulate strong cells in the tissue to activate a variety of responses, more particularly those that reduce inflammation and stop the infection. The sound waves including acoustic shock waves or pressure pulses transmit or trigger what appears to be a cellular communication throughout the entire anatomical structure, this activates a generalized cellular response at the treatment or target site, in particular, but more interestingly a systemic response in areas more removed from the wave form pattern. This is believed to be one of the reasons molecular stimulation can be conducted at threshold energies heretofore believed to be well below those commonly accepted as required. Accordingly, not only can the energy intensity be reduced but also the number of applied shock wave impulses can be lowered from several thousand to as few as one or more pulses and still yield a beneficial stimulating response if desired.

The biological model motivated the design of sources with low pressure amplitudes and energy densities. First: spherical waves generated between two tips of an electrode; and second: nearly even waves generated by generated by generalized parabolic reflectors. Third: divergent shock front characteristics are generated by an ellipsoid behind F2. Unfocused sources are preferably designed for extended two dimensional areas/volumes like skin. The unfocused sources can provide a divergent wave pattern or a nearly planar wave pattern and can be used in isolation or in combination with focused wave patterns yielding to an improved therapeutic treatment capability that is non-invasive with few if any disadvantageous contraindications. Alternatively, a focused wave emitting treatment may be used wherein the focal point extends to the desired reflexology zone or target site. In any event, the beam of acoustic waves transmitted needs to project in a large enough reflexology zone or area to stimulate or modulate the cells near the infection.

In one embodiment, the method of treatment has the steps of, locating a reflexology treatment site or zone, generating either focused shock waves or unfocused shock waves, of directing these shock waves to the treatment site; and applying a sufficient number of these shock waves to induce activation of one or more growth factor or anti-microbial peptides like LL37, thereby inducing or accelerating a modulated adjustment to induce the host cells to attack the infection.

The shock waves can be of a low peak pressure amplitude and density. Typically, the energy density values range as low as 0.000001 mJ/mm² and having a high end energy density of below 1.0 mJ/mm², preferably 0.40 mJ/mm² or less, more preferably 0.20 mJ/mm² or less. The peak pressure amplitude of the positive part of the cycle should be above 1.0 and its duration is below 1-3 microseconds.

The treatment depth can vary from the surface to the full depth of the human or animal torso and the treatment site can be defined by a much larger treatment area. The above methodology is particularly well suited for surface as well as sub-surface soft tissue treatments.

An exemplary treatment protocol could have emitted shock waves in a broad range of 0.01 mJ/mm² to 3.0 mJ/mm² and 200-2500 pulses per treatment with a treatment schedule of 1-3 weekly treatments until symptoms reduce. This can be repeated as symptoms reoccur or continue weekly as a preventative. The post medical treatment is beneficial as a pain suppressor and reduces the need for pain medications and allows less addictive medications to be used to prevent addiction. In other treatment protocols, the emitted shock waves or pressure pulses can employ as few as 1 to as high as 100,000 pulses per treatment.

The above methodology is valuable in generation of tissue, vascularization and may be used in combination with stem cell therapies as well as regeneration of tissue and vascularization.

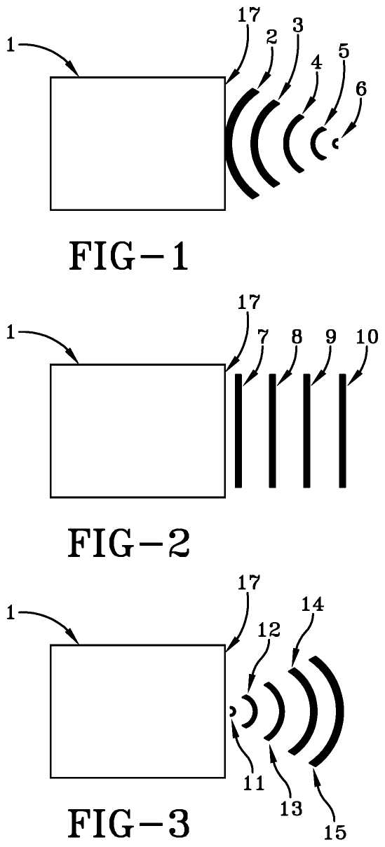

The following invention description first provides a detailed explanation of acoustic shock waves or pressure pulses, as illustrated in FIGS. 1-9. As used herein an acoustic shock wave is an asymmetric wave with an exceptionally rapid peak rise time and slower return time from the peak amplitude. Historically, these acoustic shock waves or pressure pulses were first used medically to destroy kidney stones. The wave patterns were directed to a focal point at a relatively high energy to blast the concrements into small urinary tract passable fragments.

A whole class of acoustic shock waves or pressure pulses for medical treatments were later discovered that employed low energy acoustic shock waves or pressure pulses. These low energy acoustic shock waves or pressure pulses maintained the asymmetric wave profile, but at much lower energies.

These low energy acoustic shock waves or pressure pulses advantageously could stimulate a substance without requiring a focused beam. The advantage of such an unfocused beam was the acoustic wave could be directed to pass through tissue without causing any cell rupturing which would be evidenced by a lack of a hematoma or bruising. This use of unfocused, low energy acoustic shock waves or pressure pulses provided an ability to treat a large volume of tissue virtually painlessly. Furthermore, the acoustic energy caused a short duration anesthetic sensation that effectively numbs the patient's pain over a period of days with a prolonged reduction in pain thereafter.

The use of low energy acoustic shock waves or pressure pulses that employ a focused beam has been spurred on as a viable alternative to the unfocused low energy shock waves because the focal point being of a small zone of energy has little or a small region of cell damage as the remaining portions of the wave pattern can provide a stimulating effect similar to the unfocused shock waves. Basically, the effect is the same with the users of focused waves achieving the benefits of the unfocused waves, but with a focal point of peak energy in a tiny localised region. So, for purposes of the present invention, the use of “soft waves” those defined by low energy beams will be applicable to both focused and unfocused beams of acoustic shock waves or pressure pulses.

With reference to FIGS. 1-9, a variety of schematic views of acoustic shock waves or pressure pulses are described. The following description of the proper amplitude and pressure pulse intensities of the shock waves 200 are provided below along with a description of how the shock waves actually function and have been taken from the co-pending application of the present inventors and replicated herein as described below. For the purpose of describing the shock waves 200 were used as exemplary and are intended to include all of the wave patterns discussed in the figures as possible treatment patterns.

FIG. 1 is a simplified depiction of a pressure pulse/shock wave (PP/SW) generator, such as a shock wave head, showing focusing characteristics of transmitted acoustic pressure pulses. Numeral 1 indicates the position of a generalized pressure pulse generator, which generates the pressure pulse and, via a focusing element, focuses it outside the housing to treat diseases. The affected infected tissue or organ is generally located in or near the focal point which is located in or near position 6. At position 17 a water cushion or any other kind of exit window for the acoustical energy is located.

FIG. 2 is a simplified depiction of a pressure pulse/shock wave generator, such as a shock wave head, with plane wave characteristics. Numeral 1 indicates the position of a pressure pulse generator according to the present invention, which generates a pressure pulse which is leaving the housing at the position 17, which may be a water cushion or any other kind of exit window. Somewhat even (also referred to herein as “disturbed”) wave characteristics can be generated, in case a paraboloid is used as a reflecting element, with a zone source (e.g. electrode) that is located in the focal point of the paraboloid. The waves will be transmitted into the patient's body via a coupling media such as, e.g., ultrasound gel or oil and their amplitudes will be attenuated with increasing distance from the exit window 17.

FIG. 3 is a simplified depiction of a pressure pulse shock wave generator (shock wave head) with divergent wave characteristics. The divergent wave fronts may be leaving the exit window 17 at zone 11 where the amplitude of the wave front is very high. This zone 17 could be regarded as the source zone for the pressure pulses. In FIG. 1c the pressure pulse source may be a zone source infected, that is, the pressure pulse may be generated by an electrical discharge of an electrode under water between electrode tips. However, the pressure pulse may also be generated, for example, by an explosion, referred to as a ballistic pressure pulse. The divergent characteristics of the wave front may be a consequence of the mechanical setup. The source can include radial or spherical wave generators, or linear arrays of wave generators.

This apparatus, in certain embodiments, may be adjusted/modified/or the complete shock wave head or part of it may be exchanged so that the desired and/or optimal acoustic profile such as one having wave fronts with focused, planar, nearly plane, convergent or divergent characteristics can be chosen.

A change of the wave front characteristics may, for example, be achieved by changing the distance of the exit acoustic window relative to the reflector, by changing the reflector geometry, by introducing certain lenses or by removing elements such as lenses that modify the waves produced by a pressure pulse/shock wave generating element. Exemplary pressure pulse/shock wave sources that can, for example, be exchanged for each other to allow an apparatus to generate waves having different wave front characteristics are described in detail below.

In one embodiment, mechanical elements that are exchanged to achieve a change in wave front characteristics include the primary pressure pulse generating element, the focusing element, the reflecting element, the housing and the membrane. In another embodiment, the mechanical elements further include a closed fluid volume within the housing in which the pressure pulse is formed and transmitted through the exit window.

In one embodiment, the apparatus of the present invention is used in combination therapy. Here, the characteristics of waves emitted by the apparatus are switched from, for example, focused to divergent or from divergent with lower energy density to divergent with higher energy density. Thus, effects of a pressure pulse treatment can be optimized by using waves having different characteristics and/or energy densities, respectively.

While the above described universal toolbox of the various types of acoustic shock waves or pressure pulses and types of shock wave generating heads provides versatility, the person skilled in the art will appreciate that apparatuses that produce acoustic shock waves or pressure pulses having, for one example, nearly plane characteristics, are less mechanically demanding and fulfil the requirements of many users.

As the person skilled in the art will also appreciate that embodiments shown in the drawings are independent of the generation principle and thus are valid for not only electro-hydraulic shock wave generation but also for, but not limited to, PP/SW generation based on electromagnetic, piezoceramic and ballistic principles. The pressure pulse generators may, in certain embodiments, be equipped with a water cushion that houses water which defines the path of pressure pulse waves that is, through which those waves are transmitted. In a preferred embodiment, a patient is coupled via ultrasound gel or oil to the acoustic exit window (17), which can, for example, be an acoustic transparent membrane, a water cushion, a plastic plate or a metal plate.

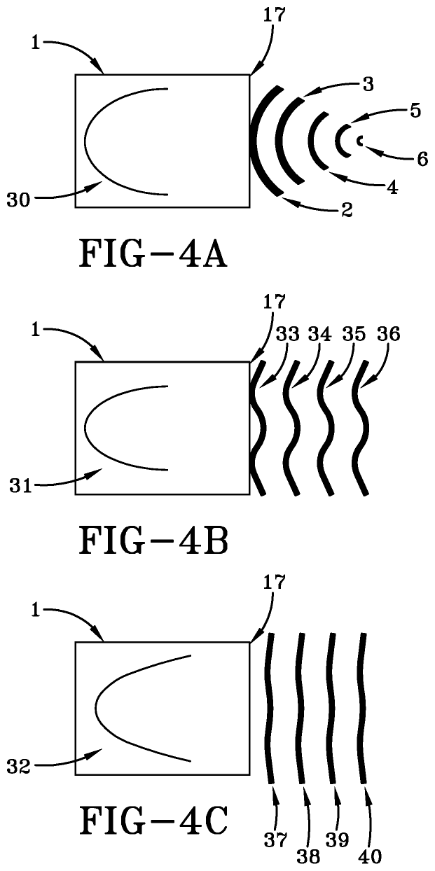

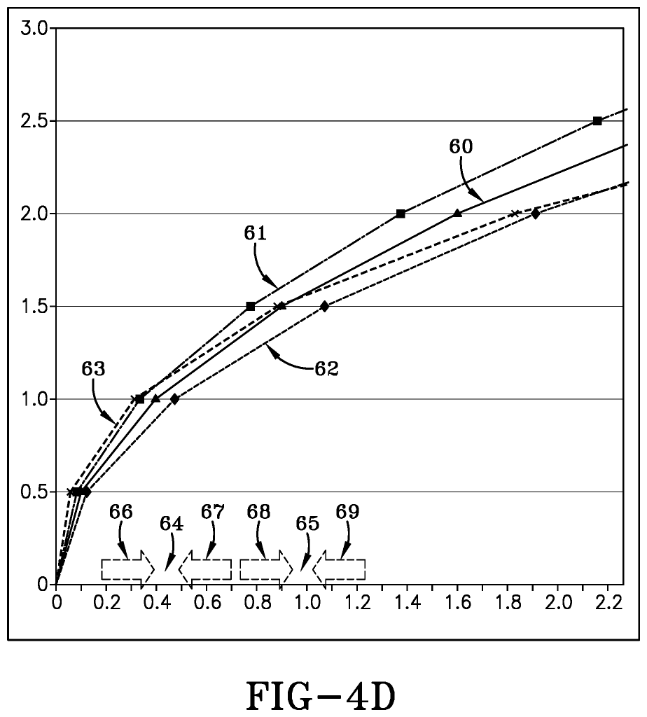

FIG. 4a is a simplified depiction of the pressure pulse/shock wave generator (shock wave head) having as focusing element an ellipsoid (30). Thus, the generated waves are focused at (6).

FIG. 4b is a simplified depiction of the pressure pulse/shock wave generator (shock wave head) having as a focusing element a paraboloid (y2=2px). Thus, the characteristics of the wave fronts generated behind the exit window (33, 34, 35, and 36) are disturbed plane (“parallel”), the disturbance resulting from phenomena ranging from electrode burn down, spark ignition spatial variation to diffraction effects. However, other phenomena might contribute to the disturbance.

FIG. 4c is a simplified depiction of the pressure pulse/shock wave generator (shock wave head) having as a focusing element a generalized paraboloid (yn=2px, with 1.2<n<2.8 and n≠2). Thus, the characteristics of the wave fronts generated behind the exit window (37, 38, 39, and 40) are, compared to the wave fronts generated by a paraboloid (y2=2px), less disturbed, that is, nearly plane (or nearly parallel or nearly even (37, 38, 39, 40)). Thus, conformational adjustments of a regular paraboloid (y2=2px) to produce a generalized paraboloid can compensate for disturbances from, e.g., electrode burn down. Thus, in a generalized paraboloid, the characteristics of the wave front may be nearly plane due to its ability to compensate for phenomena including, but not limited to, burn down of the tips of the electrode and/or for disturbances caused by diffraction at the aperture of the paraboloid. For example, in a regular paraboloid (y2=2px) with p=1.25, introduction of a new electrode may result in p being about 1.05. If an electrode is used that adjusts itself to maintain the distance between the electrode tips (“adjustable electrode”) and assuming that the electrodes burn down is 4 mm (z=4 mm), p will increase to about 1.45. To compensate for this burn down, and here the change of p, and to generate nearly plane wave fronts over the life span of an electrode, a generalized paraboloid having, for example n=1.66 or n=2.5 may be used. An adjustable electrode is, for example, disclosed in U.S. Pat. No. 6,217,531.

FIG. 4d shows sectional views of a number of paraboloids. Numeral 62 indicates a paraboloid of the shape y2=2px with p=0.9 as indicated by numeral 64 at the x axis which specifies the p/2 value (focal point of the paraboloid). Two electrode tips of a new electrode 66 (inner tip) and 67 (outer tip) are also shown in the Figure. If the electrodes are fired and the tips are burning down the position of the tips change, for example, to position 68 and 69 when using an electrode which adjusts its position to compensate for the tip burn down. In order to generate pressure pulse/shock waves having nearly plane characteristics, the paraboloid has to be corrected in its p value. The p value for the burned down electrode is indicated by 65 as p/2=1. This value, which constitutes a slight exaggeration, was chosen to allow for an easier interpretation of the Figure. The corresponding paraboloid has the shape indicated by 61, which is wider than paraboloid 62 because the value of p is increased. An average paraboloid is indicated by numeral 60 in which p=1.25 cm. A generalized paraboloid is indicated by dashed line 63 and constitutes a paraboloid having a shape between paraboloids 61 and 62. This particular generalized paraboloid was generated by choosing a value of n 2 and a p value of about 1.55 cm. The generalized paraboloid compensates for different p values that result from the electrode burn down and/or adjustment of the electrode tips.

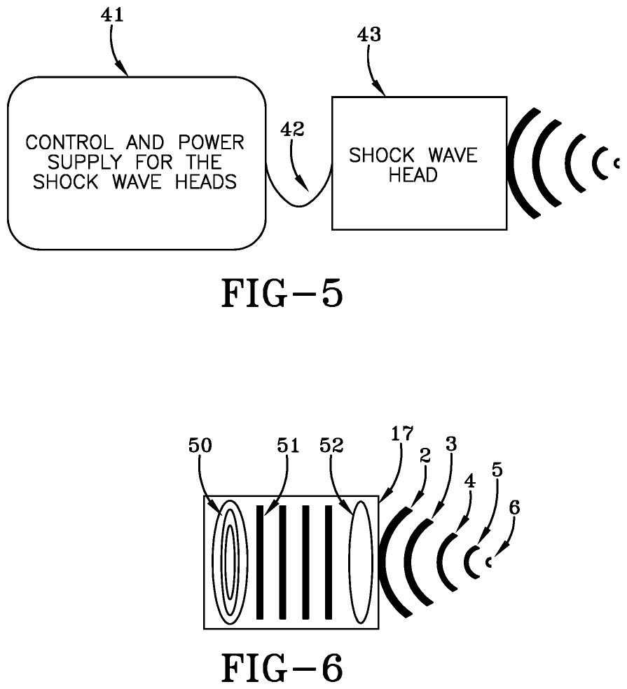

FIG. 5 is a simplified depiction of a set-up of the pressure pulse/shock wave generator (43) (shock wave head) and a control and power supply unit (41) for the shock wave head (43) connected via electrical cables (42) which may also include water hoses that can be used in the context of the present invention. However, as the person skilled in the art will appreciate, other set-ups are possible and within the scope of the present invention.

FIG. 6 is a simplified depiction of the pressure pulse/shock wave generator (shock wave head) having an electromagnetic flat coil 50 as the generating element. Because of the plane surface of the accelerated metal membrane of this pressure pulse/shock wave generating element, it emits nearly plane waves which are indicated by lines 51. In shock wave heads, an acoustic lens 52 is generally used to focus these waves. The shape of the lens might vary according to the sound velocity of the material it is made of. At the exit window 17 the focused waves emanate from the housing and converge towards focal point 6.

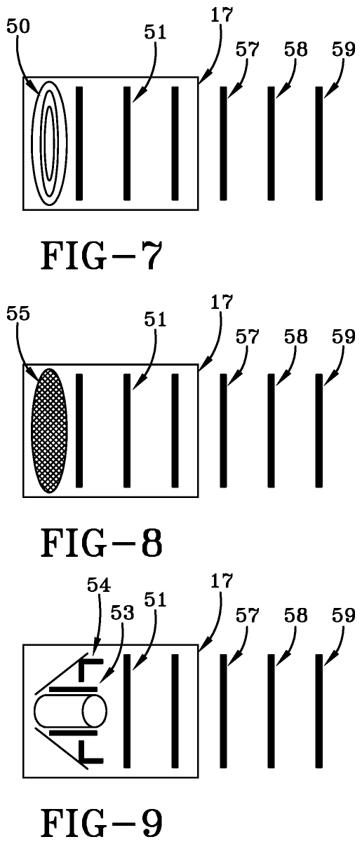

FIG. 7 is a simplified depiction of the pressure pulse/shock wave generator (shock wave head) having an electromagnetic flat coil 50 as the generating element. Because of the plane surface of the accelerated metal membrane of this generating element, it emits nearly plane waves which are indicated by lines 51. No focusing lens or reflecting lens is used to modify the characteristics of the wave fronts of these waves, thus nearly plane waves having nearly plane characteristics are leaving the housing at exit window 17.

FIG. 8 is a simplified depiction of the pressure pulse/shock wave generator (shock wave head) having a piezoceramic flat surface with piezo crystals 55 as the generating element. Because of the plane surface of this generating element, it emits nearly plane waves which are indicated by lines 51. No focusing lens or reflecting lens is used to modify the characteristics of the wave fronts of these waves, thus nearly plane waves are leaving the housing at exit window 17. Emitting surfaces having other shapes might be used, in particular curved emitting surfaces such as those shown in FIGS. 4a to 4c as well as spherical surfaces. To generate waves having nearly plane or divergent characteristics, additional reflecting elements or lenses might be used. The crystals might, alternatively, be stimulated via an electronic control circuit at different times, so that waves having plane or divergent wave characteristics can be formed even without additional reflecting elements or lenses.

FIG. 9 is a simplified depiction of the pressure pulse/shock wave generator (shock wave head) comprising a cylindrical electromagnet as a generating element 53 and a first reflector having a triangular shape to generate nearly plane waves 54 and 51. Other shapes of the reflector or additional lenses might be used to generate divergent waves as well.

In the pressure pulse or shock wave method of treating an infection within a tissue, an organ or the entire body of a host be it mechanical system or a mammal, the host system or mammal be it human or an animal with a risk of exposure to an infection or post-occurrence of such infections requires the host patient to be positioned in a convenient orientation to permit the source of the emitted waves to most directly send the waves to the target site to initiate pressure pulse or shock wave stimulation of the target area with minimal, preferably no obstructing features in the path of the emitting source or lens. Assuming the infection target area or site is within a projected area of the wave transmission, a single transmission dosage of wave energy may be used. The transmission dosage can be from a few seconds to 20 minutes or more dependent on the condition. Preferably the waves are generated from an unfocused or focused source. The unfocused waves can be divergent, planar or near planar and having a low pressure amplitude and density in the range of 0.00001 mJ/mm² to 1.0 mJ/mm² or less, most typically below 0.2 mJ/mm². The focused source preferably can use a diffusing lens or have a far-sight focus to minimize if not eliminate having the localized focus point within the tissue. Preferably the focused shock waves are used at a similarly effective low energy transmission or alternatively can be at higher energy but wherein the tissue target site is disposed pre-convergence inward of the geometric focal point of the emitted wave transmission. In treating some hard to penetrate infections, the pressure pulse more preferably is a high energy target focused wave pattern which can effectively attack the infection outer structure or barrier shield causing fractures or openings to be created to expose the colonies of microorganisms within the infection to the germicidal effects of the pressure pulses or shock waves. This emitted energy destroys the underlying microorganism's cellular membranes. In addition, the fragmentation of the infections outer barrier is then easily absorbed by or flushed out of the host. The surrounding healthy cells in the region treated are activated initiating a defense mechanism response to assist in eradication of the unwanted infection.

These shock wave energy transmissions are effective in stimulating a cellular response and can be accomplished without creating the cavitation bubbles in the tissue of the target site when employed in other than high energy focused transmissions. This effectively ensures the tissue or organ does not have to experience the sensation of hemorrhaging so common in the higher energy focused wave forms having a focal point at or within the targeted treatment site.

The present method may need precise site location and can be used in combination with such known devices as ultrasound, cat-scan or x-ray imaging if needed. The physician's general understanding of the anatomy of the patient may be sufficient to locate the target area to be treated. This is particularly true when the exposed tissue or portion of the infected body or organ is visually within the surgeon's line of sight and this permits the lens or cover of the emitting shock wave source to impinge on the affected organ or tissue directly or through a transmission enhancing gel, water or fluid medium during the pressure pulse or shock wave treatment. The treated area can withstand a far greater number of shock waves based on the selected energy level being emitted. For example, at very low energy levels the stimulation exposure can be provided over prolonged periods as much as 20 minutes if so desired. At higher energy levels the treatment duration can be shortened to less than a minute, less than a second if so desired. The limiting factor in the selected treatment dosage is to provide a stimulating stem cell activation or a cellular release or activation of the LL37 protein and VEGF and other growth factors while simultaneously germicidally attacking the infection barrier and underlying colony of microorganisms.

The underlying principle of these pressure pulse or shock wave therapy methods is to attack the infection directly and to stimulate the body's own natural healing capability. This is accomplished by deploying shock waves to stimulate strong cells in the surrounding tissue to activate a variety of responses. The acoustic shock waves transmit or trigger what appears to be a cellular communication throughout the entire anatomical structure, this activates a generalized cellular response at the treatment site, in particular, but more interestingly a systemic response in areas more removed from the wave form pattern. This is believed to be one of the reasons molecular stimulation can be conducted at threshold energies heretofore believed to be well below those commonly accepted as required. Accordingly, not only can the energy intensity be reduced in some cases, but also the number of applied shock wave impulses can be lowered from several thousand to as few as one or more pulses and still yield a beneficial stimulating response. The key is to provide at least a sufficient amount of energy to weaken the infections protective outer barrier or shield commonly found in biofilms. This weakening can be achieved by any fracture or opening that exposes the underlying colony of microorganisms.

The use of shock waves as described above achieved biological response within the cells and there appears to be a commonality in the fact that otherwise dormant cells within the tissue appear to be activated making the cell membranes more permeable to release anti-microbial peptides and absorb medications to attack infections which leads to the remarkable ability of the targeted organ or tissue to generate new growth or to regenerate weakened vascular networks increasing blood supply.

In one embodiment, the invention provides for germicidal cleaning of an infection, diseased or infected areas and for wound cleaning generally after exposure to surgical procedures.

The use of shock wave therapy requires a fundamental understanding of focused and unfocused shock waves, coupled with a more accurate biological or molecular model.

This means the physician can use these antibiotic treatments with far less adverse reactions if he combines the treatments with one or more exposures to acoustic shock waves either before introducing chemical antibiotic agents or shortly thereafter or both. This further means that the patient's recovery time should be greatly reduced because the patient treated with shock waves will have initiated a healing response that is much more aggressive than heretofore achieved without the cellular stimulation provided by pressure pulse or shock wave treatments. The current use of medications to stimulate such cellular activity is limited to absorption through the bloodstream via the blood vessels. Acoustic shock waves stimulate all the cells in the region treated activating an almost immediate cellular release of infection fighting and healing agents. Furthermore, as the use of other wise conflicting chemicals is avoided, adverse side effects can be limited to those medicaments used to destroy the infectious cells. In other words, the present invention is far more complimentary to such antibiotic treatments in that the stimulation of otherwise healthy cells will greatly limit the adverse and irreversible effects on the surrounding non-infected tissues and organs.

A further benefit of the use of acoustic shock waves is there are no known adverse indications when combined with the use of other medications or drugs. In fact, the activation of the cells exposed to shock wave treatments only enhances cellular absorption of such medication making these drugs faster acting than when compared to non stimulated cells. As a result, it is envisioned that the use of one or more medicaments prior to, during or after subjecting the patient to acoustic shock waves will be complimentary to the treatment or pre-conditioning treatment for infection exposures. It is further appreciated that certain drug therapies can be altered or modified to lower risk or adverse side effects when combined with a treatment involving acoustic shock waves as described above.

In the case wherein the patient is a victim of a infection as the result of a biological accident or a biological attack, the immediate use of shock waves shortly after exposure can be an effective tool in saving lives. The body's ability to recover is enhanced and the damaged tissue can be more quickly replaced by stimulated healthy cells which is a regenerative feature of the use of shock wave treatments.

This is particularly true in the case of infections of the skin caused by biological agents. The wounded tissue is a source of rapidly spreading infection which can lead to a complete failure of the body leading to death. The use of shock wave treatments is a valuable tool in such a case because acoustic shock waves can be provided on a virtually limitless basis as long as connected to an adequate power source. Normally supplies of medicines are limited and almost never near the area most in need. Accordingly, vehicles similar to emergency trucks used to transport patients can be equipped with shock wave generators so that in field treatments can be conducted on a wide scale quickly. This alone could greatly reduce the loss of life that would occur by delays in treatment.

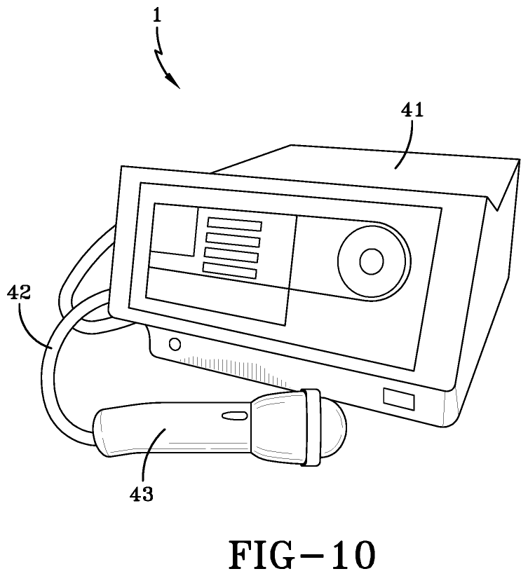

FIG. 10 shows an exemplary shock wave device generator or source 1 with a control and power supply 41 connected to a hand-held applicator shock wave head 43 via a flexible hose 42 with fluid conduits. The illustrated shock wave applicator 43 has a flexible membrane at an end of the applicator 43 which transmits the acoustic waves when coupled to the skin by using a fluid or acoustic gel. As shown, this type of applicator 43 has a hydraulic spark generator using either focused or unfocused shock waves, preferably in a low energy level, less than the range of 0.01 mJ/mm² to 0.3 mJ/mm². The flexible hose 42 is connected to a fluid supply that fills the applicator 43 and expands the flexible membrane when filled. Alternatively, a ballistic, piezoelectric or spherical acoustic shock wave device can be used to generate the desired waves.

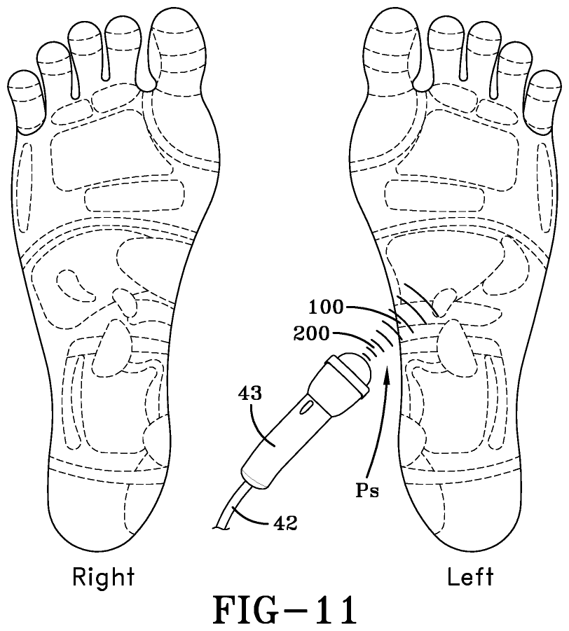

FIG. 11 is a perspective view of a foot of a patient whose reflexology zone or target 100 is being treated. A shock wave applicator head 43 is brought into contact with the skin Ps preferably an acoustic gel is used to enhance the transmission of the shock waves 200 through the skin Ps. The shock wave applicator head 43 can be hand held and manipulated across the skin Ps to drive the shock waves 200 in the direction the shock wave head 43 is zoned to activate a stimulating response through the reflexology zone 100. As illustrated, the device shown is an electrohydraulic acoustic shock wave generator, however, other devices that generate acoustic shock waves or pressure pulses can be used. Ultrasonic devices may be considered, but there is no data to support a sinusoidal wave form would work and therefore not considered as effective as the asymmetric wave generators. The acoustic shock waves or pressure pulses activate a cellular response within the reflexology treatment site. This response or stimulation causes an increase of nitric oxide and a release of a variety of growth factors such as VEGF and a release of anti-microbial peptides like LL37. As shown, the flexible membrane is protruding outward and the applicator 43 has been filled with fluid, the transmission or emission of acoustic shock waves or pressure pulses 200 is directed towards the reflexology zone 100. In order to accomplish a good transmission, it is important the flexible membrane be pressed against the patient's skin Ps and as indicated coupling gels may be used. The zone 100, as illustrated, is the reflexology zone for a bone structure which is a region of the foot located along an outside arch of each foot. By transmitting the shock waves 200 to the zone 100, is it believed that a modulation of the pain near the bone structure can be made. This modulation or adjustment can be achieved by transmitting the acoustic waves 200 at low energy directly onto the zone 100. It is believed that a single treatment of the zone 100 will achieve the desired modulation. However, repeated treatments may be administered to help maintain and control this reduced pain level. Having achieved a scheduled pattern of treatments, it is possible to achieve regulation of pain without the use of drugs or other stimulants.

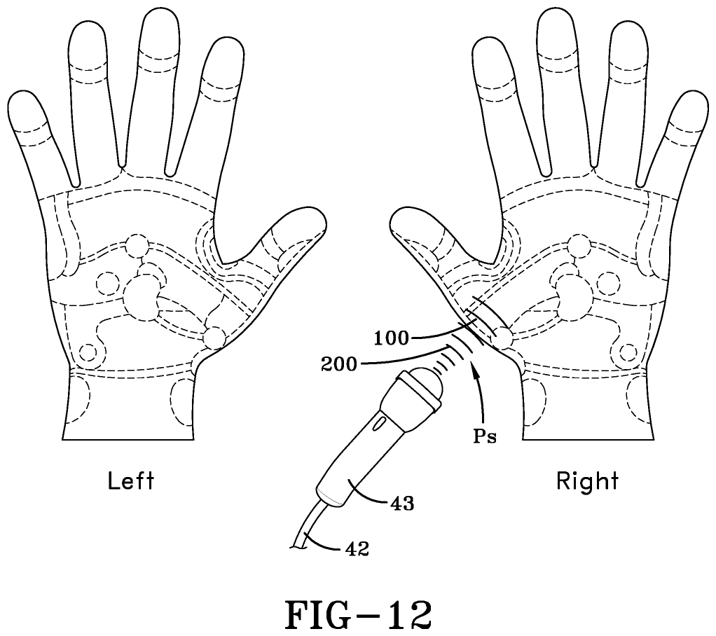

With reference to FIG. 12, a view of a hand of a patient whose reflexology zone 100 is being treated with acoustic shock waves or pressure pulses 200 is illustrated. In this illustration, it is important to note that the applicator 43 presses against the skin Ps of the hand in the reflexology zone 100 for the pancreas which is a region of the right hand in the fatty part below the index finger and a region of the left hand below the middle finger close to the wrist.

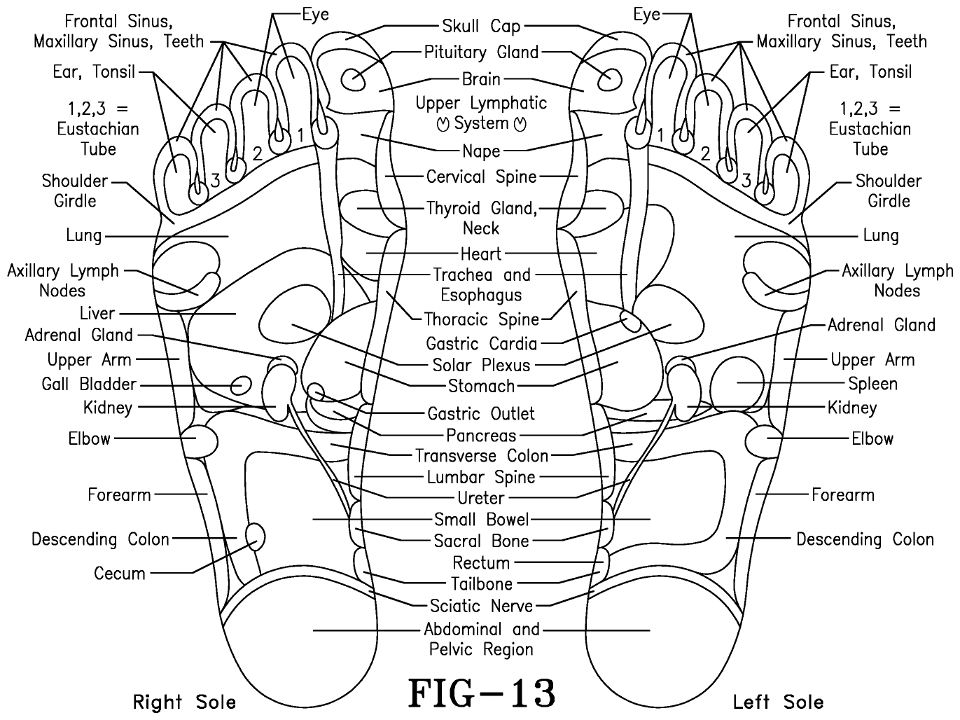

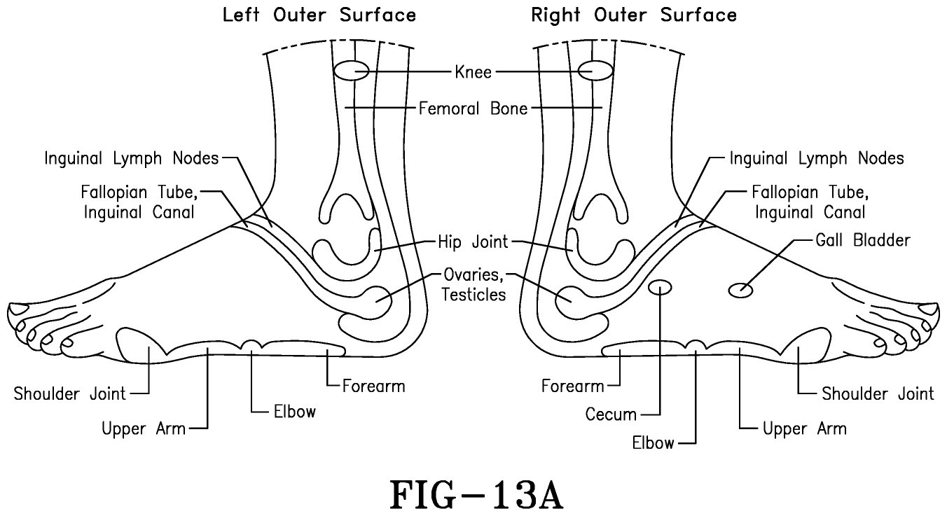

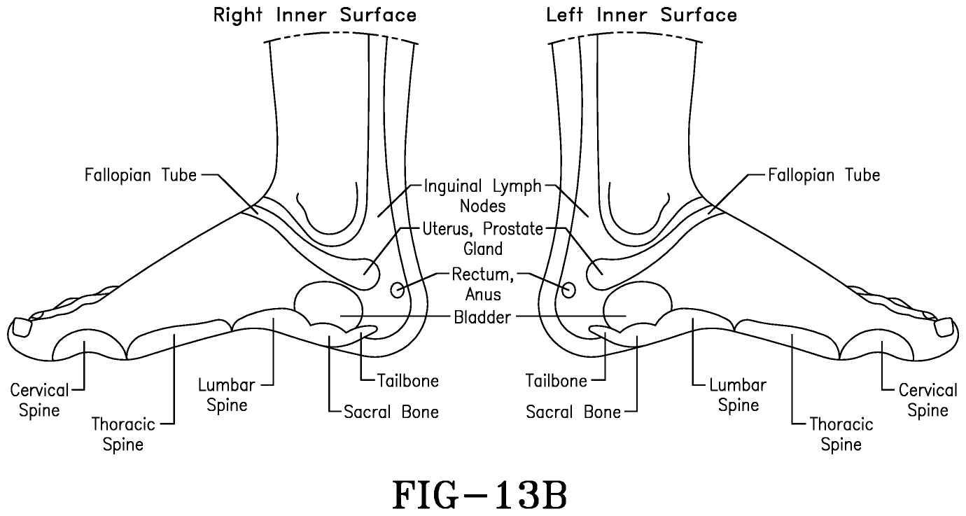

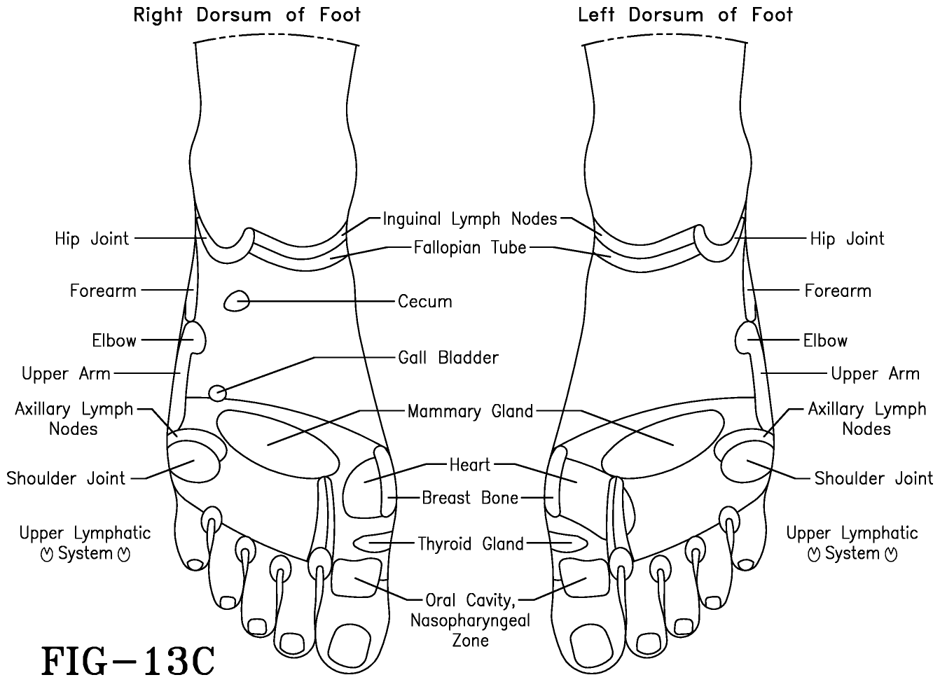

With reference to FIGS. 13-13C, reflexology foot and ankle area charts are shown detailing the various zones that correspond to organs, nerves, bones or glands of the body.

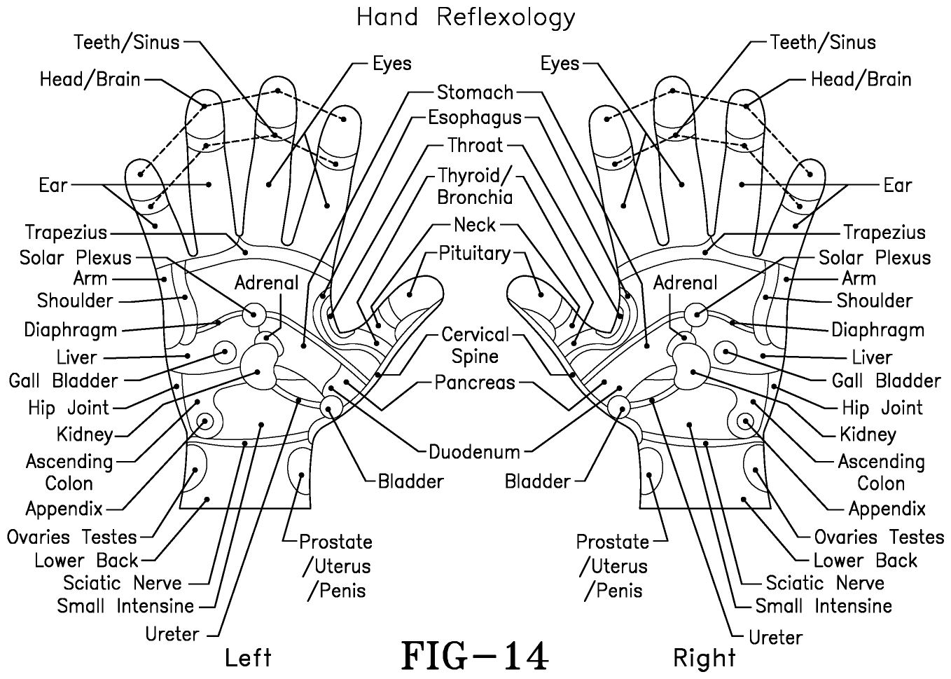

With reference to FIG. 14, a reflexology hand chart is shown detailing the various zones that correspond to organs, nerves, bones or glands of the body.

The transmission of the shock waves 200 can be of a low energy density of 0.2 mJ/mm² whether using focused or unfocused shock waves. The acoustic shock waves or pressure pulses pulse rapidly through the cells penetrating the cell membrane extremely rapidly due to the rapid rise to peak time and pass through exiting slower due to the slower return from peak amplitude. This asymmetric wave pattern rapidly compresses each cell on entry and slow decompresses the cell as it exits. This effective squeezing of each cell is believed to cause the release of growth factors such as VEGF and others and also creates nitric oxide, all beneficial to new blood vessel formation. This occurs as a transmission across the cell membranes without rupturing the native cells.

Furthermore, such acoustic shock wave forms can be used in combination with drugs, chemical treatments, irradiation therapy or even physical therapy and when so combined the stimulated cells will more rapidly assist the body's natural healing response and thus overcomes the otherwise potentially tissue damaging effects of these complimentary procedures.

The present invention provides an apparatus for an effective treatment of indications, which benefit from high or low energy pressure pulse/shock waves having focused or unfocused, nearly plane, convergent or even divergent characteristics. With an unfocused wave having nearly plane, plane, convergent wave characteristic or even divergent wave characteristics, the energy density of the wave may be or may be adjusted to be so low that side effects including pain are very minor or even do not exist at all.

In certain embodiments, the apparatus of the present invention is able to produce waves having energy density values that are below 0.1 mJ/mm² or even as low as 0.000001 mJ/mm². In a preferred embodiment, those low end values range between 0.1-0.001 mJ/mm². With these low energy densities, side effects are reduced and the dose application is much more uniform. Additionally, the possibility of harming surface tissue is reduced when using an apparatus of the present invention that generates unfocused waves having planar, nearly plane, convergent or divergent characteristics and larger transmission areas compared to apparatuses using a focused shock wave source that need to be moved around to cover the affected area. The apparatus of the present invention also may allow the user to make more precise energy density adjustments than an apparatus generating only focused shock waves, which is generally limited in terms of lowering the energy output. Nevertheless, in some cases the first use of a high energy focused shock wave targeting a treatment zone may be the best approach followed by a transmission of lower energy unfocused wave patterns.

In the use of reflexology zones as the indirect pathway or gate to stop infection and cure diseases/disorders and/or control pain response, the present invention has actual empirical data showing the effectiveness in the zone directed to a bone. It is therefore further believed that similar modulation and beneficial adjustment can be achieved at other reflexology zones for stimulating, modulating or adjusting other glands, bones, nerves or organs such as the liver, kidney or any of those indicated in FIG. 13 for the foot zones and FIG. 14 for the hand zones. It is further believed that the hybrid Eastern medical acupuncture treatments or massages historically used are far less effective and less reliable than the results achieved by the deeper tissue penetrating transmission that are achieved by acoustic shock wave therapy applied to these reflexology zones.

Included in treatments for infections are all auto immune indications/disorders as well as disorders of chronic local and systemic inflammation, congestive heart or lung failure. Mechanism is reduction of any systemic inflammation, drastically lower the white blood cell count and causing the body to stop attacking itself. Treatments can be applied weekly to hands and feet, maximum 2500 each, treating the entire foot or hand, focusing on those painful zones until the pain disappears or decreases substantially, preferably treating for 4 weeks or less. Also, if patient has heart inflammation/congestive heart failure, focusing on the known reflexology zones for hearts and lungs. These spots may be painful at first.

When treating the hands and feet and noting the painful spots, one can locate areas on the known reflexology zone charts to diagnose weaknesses, or injuries in the body at each corresponding zone.

Another treatment includes treating for high or low numbers of eosinophils. The most common causes of a high number of eosinophils (called eosinophilia or hypereosinophilia) are Allergic disorders, Infections by parasites, or Certain cancers. Allergic disorders, including asthma, allergic rhinitis, and atopic dermatitis, often increase the number of eosinophils. Many parasites, particularly ones that invade tissue, cause eosinophilia. Cancers that cause eosinophilia include Hodgkin lymphoma, Leukemia, and certain myeloproliferative disorders.

If the number of eosinophils is only slightly elevated, people usually do not have symptoms, and the high number of eosinophils in the blood is only discovered when a complete blood count is done for other reasons. However, sometimes, particularly when the number of eosinophils is very high, the increased number of eosinophils inflame tissues and cause organ damage. The heart, lungs, skin, and nervous system are most often affected, but any organ can be damaged. Symptoms are related to the organ affected. For example, people may have a rash when the skin is affected, wheezing and shortness of breath when the lungs are affected, shortness of breath and fatigue (symptoms of heart failure) when the heart is affected, or throat and stomach pain when the esophagus or stomach is affected. Accordingly, eosinophilic disorders are diagnosed according to the location where the levels of eosinophils are elevated: Eosinophilic pneumonia (lungs), Eosinophilic cardiomyopathy (heart), Eosinophilic esophagitis (esophagus), Eosinophilic gastritis (stomach), Eosinophilic enteritis (small intestine), Crohns, Rheumatoid arthritis, MS Multiple Sclerosis, IBS irritable bowel syndrome, Primary myelofibrosis, Polycythemia vera, Thrombocythemia, Chronic Myelogenous Leukemia, CML—Chronic Myelocytic Leukemia; Chronic Myeloid Leukemia; Chronic Granulocytic Leukemia, Sickle cell anemia. Treating the feet and hands with 1000 shocks a piece cures the symptoms in these diseases. Currently there are no known cures for any of these disorders. Treating the patient one time with unfocused wave therapy cures the symptoms of these disease pathologies. In theory the body turns off the eosinophilis in the inventors' opinion.

Another treatment includes treating for Nocturia (excessive urination at night). Treatment reduces swollen prostate, strengthens bladder, reduces inflammation, drastically reducing night time urination in a matter of a few weeks, preferably with four weekly treatments. Nocturia and BPH (benign prostatectomy hyperplasia), incontinence and interstitial cystitis in women, plus vaginal rejuvenation have high treatment demand Treatment strengthens muscles, tissue in pelvic floor and the vagina by increasing blood supply, reducing inflammation and recruitment of stem cells. Treatment in males, treating prostate, shrinks prostate, increase urine flow, and need to urinate less frequently. Bladder lies over prostate, the tri-gone area is in between that is what is being treated, also the bladder neck. The trigone (a.k.a. vesical trigone) is a smooth triangular region of the internal urinary bladder formed by the two ureteric orifices and the internal urethral orifice. The area is very sensitive to expansion and once stretched to a certain degree, the urinary bladder signals the brain of its need to empty. The signals become stronger as the bladder continues to fill. This tri-gone area can be treated in women also for Trigonitis, the condition when the vesical trigone area of the urinary bladder gets inflamed. The cells in the lower bladder partly change into another kind of cell though the changes are not cancerous in nature. Vesical trigone is the triangular region of the bladder, which is bound by the ureteral orifices and the urethral sphincter. It is a smooth and flat sensitive region and if the bladder fills up, it expands too. If the vesical region expands, the bladder is required to be emptied. Trigonitis is mostly found in women of childbearing age and men develop it occasionally.

Treatment of the prostate can be targeted through the perineum, or the pelvic opening at the base of the penis. Preferably, it can be targeted through the rectum (picture finger prostate exam). One would be side firing, and utilize an integrated ultrasound softwave technology that will allow visualization of the target of the sound waves on the ultrasound devices screen (prostate). Picture crosshairs on the screen that will show when the probe is zoned in the correct direction and the correct distance. Both BPH and Nocturia and increased urine flow rates are cured by the device, or any other indication that benefits from a less inflamed, smaller prostate, and nerves regenerated. Treatment also strengthen the bladder, bladder neck and sphincter. 500-2000 shocks have been applied in 3-6 treatments at energy densities between 0.04 mJ/mm² to 0.14 mJ/mm². In treatments, success rates on the first 20 patients exceeds 75%.

For interstitial cystitis, and vaginal tightening/rejuvenation, treatment is very similar to the above protocol. The pelvic floor, bladder neck, sphincter, and bladder can all be targeted directed through the skin surface, above and below the vaginal opening. The same number of shocks and ranges as above. Another type of probe that does not have a lens but fires a spherical wave targeting all of the walls of the vagina at the same time could be used. This version does not have to be incorporated with the ultrasound imaging as above although that probe would work. Tissue is strengthened, inflammation reduced, nerves regenerated, and stem cells recruited and activated. All acoustic waves, focused and unfocused, spherical, radial, ballistic, etc. could be used for treatments.

The data from the first 14 patient interviews showed some interesting statistics: For ED: The average improvement of the 14 patients was 48%, 13/14 showed some improvement. Counting only those who showed improvement, the average improvement was 52%, 9/14 showed at least a 50% improvement, Nocturia: The overall average improvement of the 10 patients who complained of Nocturia was 46%. Nocturia was defined as those patients who urinated at least 2 times per night for the sake of this study. 7/10 showed improvement, all over 50%, Counting only those who improved (7/10), the average improvement was 65%, Improved Urine Flow: The average improvement of the 10 patients who cited poor urine flow was 51%, 7/10 patients showed some improvement, all greater than 50%, Counting only those patient who improved, the average improvement was 72%, It is important to note that Nocturia and increased urine flows were not the original targets. Initially, a maximum energy density of 0.1 mJ was not thought high enough to reach the prostate/bladder interface to affect these symptoms via the perineum, surprisingly, the low energy density did work. These results were not intended or anticipated. Future results should improve as we target these critical areas and new results confirm this. These results are amazing. The fact that 3 patients showed no improvement might be explained that the random nature of the perineum treatment did not direct enough energy to the prostate and bladder.

Final summary: No patients complained of pain. All patients expressed interest in additional treatments. 12/14 patients had at least 1 improvement of symptom scores of at least 50% when you include ED and Nocturia. 1 patient had a 100% improvement in ED, 83% reduction in the number of times urinating each night (6 to 1), and a 90% increase in urine flow.

Reflexology methods of treating infections using both feet and hands to generate total wellness, and more specifically this treatment reduces inflammation systemically. No device can do that. This reduction in systemic inflammation cures all auto immune disorders. A body stops attacking itself. Reflexology treats a specific part of the foot to treat a specific infected target/organ. Treating all of the zones resets a body's overall wellness.

In the application of reflexology treatment of the present invention, pathologic tissue can be targeted directly and in combination with the named identifiable reflexology zones which is believed to be the best possible therapy. Shock wave or pressure pulse treatment can cure any part of the body by treating using the combination of directly treating the tissue and also treating the appropriate reflexology zone. One can also diagnose bodily deficiencies/injuries/pathologies by pulsing all of the reflexology zones of the hands and feet and noting the painful areas. These painful areas will correspond to a pathologic location in the body. The heart zone will hurt in a congestive heart failure patient. Continue to treat this painful spot until inflammation is gone and the appropriate biologic cascade has been activated in the heart.

There are two main groups of adrenoreceptors, α and β, with 9 subtypes in total: α are divided to α1 (a Gq coupled receptor) and α2 (a Gi coupled receptor); α1 has 3 subtypes: α1A, α1B and α1D; α2 has 3 subtypes: α2A, α2B and α2C; β are divided to β1, β2 and β3. All 3 are coupled to Gs proteins, but β2 and β3 also couple to Gi. Gi and Gs are linked to adenylyl cyclase. Agonist binding thus causes a rise in the intracellular concentration of the second messenger cAMP. Gi inhibits the production of cAMP. Downstream effectors of cAMP include cAMP-dependent protein kinase (PKA), which mediates some of the intracellular events following hormone binding. Epinephrine (adrenaline) reacts with both α- and β-adrenoreceptors, causing vasoconstriction and vasodilation, respectively. Although a receptors are less sensitive to epinephrine, when activated at pharmacologic doses, they override the vasodilation mediated by β-adrenoreceptors because there are more peripheral al receptors than β-adrenoreceptors. The result is that high levels of circulating epinephrine cause vasoconstriction. However, the opposite is true in the coronary arteries, where β2 response is greater than that of α1, resulting in overall dilation with increased sympathetic stimulation. At lower levels of circulating epinephrine (physiologic epinephrine secretion), β-adrenoreceptor stimulation dominates since epinephrine has a higher affinity for the β2 adrenoreceptor than the α1 adrenoreceptor, producing vasodilation followed by decrease of peripheral vascular resistance. Smooth muscle behavior is variable depending on anatomical location. One important note is the differential effects of increased cAMP in smooth muscle compared to cardiac muscle. Increased cAMP will promote relaxation in smooth muscle, while promoting increased contractility and pulse rate in cardiac muscle.

α receptors have actions in common, but also individual effects. Common or still receptor unspecified actions include: vasoconstriction and decreased motility of smooth muscle in gastrointestinal tract. Subtype unspecific a agonists can be used to treat rhinitis, they decrease mucus secretion. Subtype unspecific a antagonists can be used to treat pheochromocytoma, they decrease vasoconstriction caused by norepinephrine.

α1-adrenoreceptors are members of the Gq protein-coupled receptor superfamily Upon activation, a heterotrimeric G protein, Gq, activates phospholipase C (PLC). The PLC cleaves phosphatidylinositol 4,5-bisphosphate (PIP2), which in turn causes an increase in inositol triphosphate (IP3) and diacylglycerol (DAG). The former interacts with calcium channels of endoplasmic and sarcoplasmic reticulum, thus changing the calcium content in a cell. This triggers all other effects, including a prominent slow after depolarizing current (sADP) in neurons. Actions of the al receptor mainly involve smooth muscle contraction. It causes vasoconstriction in many blood vessels, including those of the skin, gastrointestinal system, kidney, renal artery, and brain. Other areas of smooth muscle contraction are: ureter, vas deferens, hair (arrector pili muscles), uterus (when pregnant), urethral sphincter, urothelium and lamina propria, bronchioles (although minor relative to the relaxing effect of β2 receptor on bronchioles), blood vessels of ciliary body (stimulation causes mydriasis). Actions also include glycogenolysis and gluconeogenesis from adipose tissue and liver; secretion from sweat glands and Na+ reabsorption from kidney. α1 antagonists can be used to treat: hypertension, they decrease blood pressure by decreasing peripheral vasoconstriction and benign prostate hyperplasia, they relax smooth muscles within the prostate thus easing urination.

The α2 receptor couples to the Gi/o protein. It is a presynaptic receptor, causing negative feedback on, for example, norepinephrine (NE). When NE is released into the synapse, it feeds back on the α2 receptor, causing less NE release from the presynaptic neuron. This decreases the effect of NE. There are also α2 receptors on the nerve terminal membrane of the post-synaptic adrenergic neuron. Actions of the α2 receptor include: decreased insulin release from the pancreas, increased glucagon release from the pancreas, contraction of sphincters of the GI-tract, negative feedback in the neuronal synapses—presynaptic inhibition of norepinephrine release in CNS, increased platelet aggregation (increased blood clotting tendency), decreases peripheral vascular resistance. α2 agonists can be used to treat: hypertension, they decrease blood pressure raising actions of the sympathetic nervous system, impotence, they relax penile smooth muscles and ease blood flow and depression, they enhance mood by increasing norepinephrine secretion.

Subtype unspecific β agonists can be used to treat: heart failure, they increase cardiac output acutely in an emergency, circulatory shock, they increase cardiac output thus redistributing blood volume, and anaphylaxis-bronchodilation. Subtype unspecific β antagonists, beta blockers, can be used to treat: heart arrhythmia, they decrease the output of sinus node thus stabilizing heart function, coronary artery disease, they reduce heart rate and hence increasing oxygen supply, heart failure, they prevent sudden death related to this condition which is often caused by ischemias or arrhythmias, hyperthyroidism, they reduce peripheral sympathetic hyperresponsiveness, migraine, they reduce number of attacks, stage fright, they reduce tachycardia and tremor, glaucoma, they reduce intraocular pressure.

Actions of the β1 receptor include: increase cardiac output by increasing heart rate (positive chronotropic effect), conduction velocity (positive dromotropic effect), stroke volume (by enhancing contractility-positive inotropic effect), and rate of relaxation of the myocardium, by increasing calcium ion sequestration rate (positive lusitropic effect), which aids in increasing heart rate; increase renin secretion from juxtaglomerular cells of the kidney and increase ghrelin secretion from the stomach.

β2 adrenoreceptor (PDB: 2rh1) stimulates cells to increase energy production and utilization. Actions of the β2 receptor include: smooth muscle relaxation throughout many areas the body, e.g. in bronchi (bronchodilation, see salbutamol), GI tract (decreased motility), veins (vasodilation of blood vessels), especially those to skeletal muscle (although this vasodilator effect of norepinephrine is relatively minor and overwhelmed by a adrenoceptor-mediated vasoconstriction), lipolysis in adipose tissue, anabolism in skeletal muscle, relax non-pregnant uterus, relax detrusor urinae muscle of bladder wall, dilate arteries to skeletal muscle, glycogenolysis and gluconeogenesis, stimulates insulin secretion, contract sphincters of GI tract, thickened secretions from salivary glands, inhibit histamine-release from mast cells, increase renin secretion from kidney, and involved in brain-immune communication. β2 agonists can be used to treat: asthma and COPD, they reduce bronchial smooth muscle contraction thus dilating the bronchus, hyperkalemia, they increase cellular potassium intake, and preterm birth, they reduce uterine smooth muscle contractions.

Actions of the β3 receptor include: increase of lipolysis in adipose tissue. β3 agonists could theoretically be used as weight-loss drugs, but are limited by the side effect of tremors.

Shock wave or pressure pulse treatment can modulate alpha 1 and 2, beta, and other adrenergic receptors by directly targeting the tissue AND by the stimulation of the reflexology zones. For example, by targeting the hearts reflexology zones you can modulate alpha receptors in the heart. Shock wave or pressure pulse treatment can recruit, activate and differentiate stem cells by directly targeting the pathologic tissue or by targeting the pertinent reflexology zones or preferably by doing both in combination. This is the same for modulating inflammation locally by the direct targeting or modulating SYSTEMIC inflammation by treating any or all of the reflexology zones.

A new aspect emerged in an experimental work from Würzburg, in which old rats were treated with appropriately aged penis tissue. As a result, a regeneration and rejuvenation of the tissue was confirmed. However, in addition to the known factors, alpha 1 and alpha 2 adrenergic receptors were also measured, and it was found that these factors had changed significantly. This means, according to the mechanisms of this factor, that also psychogenic factors of the patients benefit from the treatment. Thus, a new spectrum of therapies using the present invention could result scientifically justified on the mechanism of alpha1 and alpha2 changes and modulation. Not only the treatment of the psychogener ED, erectile dysfunction, but also the treatment of the longlife PE could be effectively treated. This similarly would foreseeably assist in overcoming the occurrence of premature ejaculation, PE, by providing an ability to modulate these adrenergic receptors leading to a new found hope for those suffering from either ED or PE.

Pressure pulse or acoustic shock wave treatment can result in reduced levels of PSA possibly caused by the anti-inflammatory effect of the treatments. The application of acoustic shock waves can not only reduce the risk of/prevent cancer and disease in any targeted (at risk) tissue or organs. Antigens (inflammation) in the prostate is reduced and this reduces disease/cancer risks. Inflammation of the heart is reduced reducing risk of heart disease. This heart/valve treatment stops the growth of calcification in the aorta and will causes the calcification to be reabsorbed. PSA levels in the prostate as well as the inflammatory markers in the heart can be decreased when the heart is targeted non-invasively.

Pressure pulse or acoustic shock wave treatment after a snakebite prevents spread of necrotic tissue.

It will be appreciated that the apparatuses and processes of the present invention can have a variety of embodiments, only a few of which are disclosed herein. It will be apparent to the artisan that other embodiments exist and do not depart from the spirit of the invention. Thus, the described embodiments are illustrative and should not be construed as restrictive.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. A method of treating a patient having an infection from bacteria or molds or fungi or virus by destroying bacteria or molds or fungi or virus comprising the step of:
directing one or more sound wave treatments into the patient to destroy bacteria or molds or fungi or virus.

2. The method of claim 1 wherein the sound wave treatments cause an improved blood supply, a disruption of cellular membranes and a cellular communication causing the patient's cells to identify and attack the bacteria, mold fungi or virus and further causes recruiting or stimulating an increase in anti-microbial peptides.

3. The method of claim 1 further comprises the step of:
administering medications to the patient including, but not limited to anti-viral medications, antibiotics, anti-fungal medications or anti-mold medications, wherein the sound wave treatment extends the useful life of the medications.

4. The method of claim 3 wherein the sound wave treatments increase the permeability of the patient's cell membranes allowing an increase in releasing anti-microbial peptides and inflow of the medications into the cells while increasing the blood supply toward the infection.

5. The method of claim 3 wherein the sound wave treatment is provided either prior to, during or after administering medications or any combination thereof.

6. The method of claim 5 wherein the infection's resistance to medications is reduced by the sound wave treatments.

7. The method of claim 5 wherein the medications effectiveness against the infection is enhanced by the sound wave treatments.

8. The method of claim 5 wherein the dosages or strength of the medications can be reduced when used in combination with the sound wave treatments.

9. The method of claim 1 wherein the sound waves are acoustic shock waves.

10. The method of claim 9 wherein the acoustic shock waves are focused or non-focused, convergent, divergent, planar or nearly planar, radial or spherical, shaped or otherwise reflected.

11. The method of claim 10 wherein the sound wave treatments are emitted by a generator.

12. The method of claim 11 wherein the generator is one of a radial, a spherical, a ballistic, a linear, a piezoelectric, or an electrohydraulic generator.

13. The method of claim 1 wherein the sound wave treatments can be administered with or without cavitation.

14. The method of claim 1 wherein the sound wave treatments can be administered with or without some cellular destruction and with or without a sensation of pain.

15. The method of treating a patient diagnosed with one or more infections of a microbial or viral source, the infections causing at least localized inflammation, the method comprises the steps of:
locating a region or location of the infection;
activating a pressure pulse or acoustic shock wave generating source; and
emitting pressure pulses or acoustic shock waves and directing the pressure pulses or acoustic shock waves to impinge the inflammation directly or by indirectly impinging a reflexology zone to destroy, fracture, fragment or otherwise open the microbial or viral source to eradicate the source and reduce the inflammation.

16. The method of claim 15 further comprises the step of:
stimulating cells of a host to initiate a cellular response within the host when the host is a living being with organs and tissues having a cellular structure, the stimulated cells assist in absorbing or otherwise eradicating the microbial or viral source. fragment or otherwise open the microbial or viral source to eradicate the source and reduce the inflammation.

17. The method of claim 15 wherein the emitted pressure pulses or acoustic shock waves impinge the underlying bacterial or viral organisms destroying or rupturing their outer membranes to germicidally kill the organisms.

18. The method of claim 15 further comprises the step of:
administering one or more drugs, antibiotics or other medication to the host.

19. The method of claim 15 further comprises the step of:
surgically exposing the region or location of the infection.

20. The method of treatment of claim 15 wherein the emitted pressure pulses or acoustic shock waves are focused or non-focused waves of convergent, divergent, planar or near planar pattern.

21. The method of treatment of claim 15 wherein the emitted pressure pulses or acoustic shock waves are convergent having one or more geometric focal volumes of points at a distance of at least X from the generator or source, the method further comprising positioning the organ at a distance at or less than the distance X from the source.

22. The method of treatment of claim 15 further comprises the step of:
administering one or more medications prior, during or after subjecting the patient to pressure pulses or acoustic shock waves.

23. The method of treatment of claim 15 further comprises the step of:
subjecting a tissue or organ to a surgical procedure to remove some or all of an infection growth.

24. The method of claim 15 wherein the region or location is part of a system including the cardiovascular, urological, reproductive, digestive, intestinal, neurological or periodontal.

25. The method of claim 15 wherein the pressure pulses or acoustic shock waves cause an upregulation or increase of antimicrobial peptides LL37

26. The method of claim 15 wherein the infection is generally non-responsive to medications.

27. A method of treating a patient having inflammation comprising the step of:
directing one or more sound wave treatments into the patient toward the inflammation.

28. The method of claim 27 wherein the sound wave treatments cause an improved blood supply, a disruption of cellular membranes and a cellular communication causing the patient's cells to identify the source of the inflammation and to reduce or eliminate the inflammation.

29. The method of claim 27 further comprises the step of:
administering medications to the patient including, but not limited to anti-viral medications, antibiotics, anti-fungal medications or anti-mold medications, wherein the sound wave treatment extends the useful life of the medications.

30. The method of claim 29 wherein the sound wave treatments increase the permeability of the patient's cell membranes allowing an increase in releasing anti-microbial peptides and inflow of the medications into the cells while increasing the blood supply toward the inflammation.

31. The method of claim 29 wherein the sound wave treatment is provided either prior to, during or after administering medications or any combination thereof.

32. The method of claim 31 wherein the inflammation's resistance to medications is reduced by the sound wave treatments.

33. The method of claim 31 wherein the medications effectiveness against the inflammation is enhanced by the sound wave treatments.

34. The method of claim 31 wherein the dosages or strength of the medications can be reduced when used in combination with the sound wave treatments.

35. The method of claim 27 wherein the sound waves are acoustic shock waves.

36. The method of claim 35 wherein the acoustic shock waves are focused or non-focused, convergent, divergent, planar or nearly planar, radial or spherical, shaped or otherwise reflected.

37. The method of claim 36 wherein the sound wave treatments are emitted by a generator.

38. The method of claim 37 wherein the generator is one of a radial, a spherical, a ballistic, a linear, a piezoelectric, or an electrohydraulic generator.

39. The method of claim 27 wherein the sound wave treatments can be administered with or without cavitation.

40. The method of claim 27 wherein the sound wave treatments can be administered with or without some cellular destruction and with or without a sensation of pain.
